(19) United States
(12) Patent Application Publication
(10) Pub. No.: US 2026/0335589 A1
(43) Pub. Date: Oct. 29, 2026

(54) PACKAGING FOR MEDICAL DEVICE

(71) Applicant: Edwards Lifesciences Corporation, Irvine, CA (US)

(72) Inventors: Laura Elizabeth Wasson, Silverado, CA (US); Tito Manuel Cairo Nunez, Tustin, CA (US); Robert MacCarthy, Irvine, CA (US); Sameer Dinmanishanker Upadhyaya, Irvine, CA (US); Kyle Takashi Minamide, Rancho Santa Margarita, CA (US); James Ryan Head, Santa Ana, CA (US); Chloe Jean Watters, East Lansing, MI (US)

(21) Appl. No.: 19/713,441

(22) Filed: Jun. 18, 2026

Related U.S. Application Data
(63) Continuation of application No. PCT/US2024/058973, filed on Dec. 6, 2024.
(60) Provisional application No. 63/613,893, filed on Dec. 22, 2023.

(51) Int. Cl. A61M 25/00

(52) U.S. Cl. CPC A61M 25/002

(57) ABSTRACT

This disclosure relates to a packaging for medical devices and, more particularly, to a method and system for sterile packaging of an elongated surgical device. The packaging for an elongated device may comprise a tube comprising a tube space therein; a device holder configured to releasably hold a proximal portion of the elongated device, a removable cap; and an end cap disposed at a distal end of the tube. When the tube, the device holder, and the removable cap are assembled, the device holder is configured to hold the proximal portion of the elongated device such that the elongated device is suspended and does not touch the internal surface of the tube.

### TECHNICAL FIELD

This application is a continuation of PCT Patent Application No. PCT/US2024/058973, filed on Dec. 6, 2024, which claims the benefit of U.S. Provisional Patent Application No. 63/613,893, filed on Dec. 22, 2023, each of these applications being incorporated herein by reference in its entirety.

### TECHNICAL FIELD

This disclosure relates to packaging for medical devices and, more particularly, to a method and system for sterile packaging of a surgical device.

### BACKGROUND

The packaging of surgical devices has presented numerous challenges. Elongated surgical devices, such as catheters, are conventionally packaged in elongated containers for shipping and storage prior to use in the operating theater. For more delicate surgical devices, a tray system or other containment system may be used to provide additional support features; however, these packaging systems may be bulkier or have multiple layers or materials to provide adequate protection.

In view of these limitations, opportunities for alternative packaging for such elongated surgical devices arise that will provide better protection and simplified field deployment.

### SUMMARY

In one aspect, a packaging for an elongated device is provided. The packaging comprises: a tube comprising a tube space therein; a device holder configured to releasably hold a proximal portion of the elongated device, wherein the device holder comprises: a tube contact portion extending from a distal end of the device holder, the tube contact portion configured to be placed within the tube space and touch at least a portion of an internal surface of the tube, a handle portion extending from a proximal end of the device holder, a ledge between the tube contact portion and the handle portion, the ledge configured to rest on a proximal end of the tube such that the tube contact portion can be inserted into the tube space while the handle portion remains outside the tube, and a housing cavity configured to house the proximal portion of the elongated device; a removable cap having an internal space configured to house the handle portion of the device holder; and an end cap disposed at a distal end of the tube; wherein the removable cap is configured to be reversibly engaged with the proximal end of the tube; and wherein, when the tube, the device holder, and the removable cap are assembled, the device holder is configured to hold the proximal portion of the elongated device such that the elongated device is suspended and does not touch the internal surface of the tube.

In some embodiments, the removable cap and the proximal end of the tube comprise threads. In some embodiments, the removable cap is internally threaded; and the proximal end of the tube is externally threaded. In some embodiments, the removable cap and the tube are configured to form a microorganism-tight seal, such that microorganisms are inhibited from entering the tube space. In some embodiments, the removable cap and the tube are configured to form a tortuous path, such that gas is allowed to ingress to or egress from the tube space via the tortuous path, while microorganisms are inhibited from entering the tube space. In some embodiments, the packaging further comprises a gasket or an O-ring. In some embodiments, the device holder has a plurality of divisions that are configured to be separated to expose the housing cavity. In some embodiments, the device holder further comprises a hinge. In some embodiments, the device holder further comprises a snap-fit mechanism, such that the plurality of divisions of the device holder can be kept assembled. In some embodiments, the plurality of divisions of the device holder are configured to be separated by sliding. In some embodiments, the device holder is configured to contact at least a portion of an internal surface of the removable cap. In some embodiments, the device holder has a length; and the tube contact portion extends at least half of the length of the device holder. In some embodiments, the ledge is configured to prevent the handle portion from entering the tube space. In some embodiments, the housing cavity substantially conforms to a shape of the elongated device. In some embodiments, the tube contact portion has a length which is at least 20% of the length of the tube. In some embodiments, the tube contact portion has a length which is at least 1.5 times the diameter of the elongated device, which may be a surgical device.

Each feature or concept outlined above is independent and can be combined with other features or concepts outlined above or with any other feature or concept disclosed in this application.

In one aspect, a kit comprising the packaging described in preceding paragraphs, and an elongated surgical device is provided.

In some embodiments, the surgical device is a catheter device. In some embodiments, the surgical device does not touch the internal surface of the tube of the packaging.

In one aspect, a method of deploying an elongated surgical device is provided. The method comprises: providing the elongated surgical device contained in a packaging, wherein the packaging comprises: a tube comprising a tube space therein; a device holder releasably holding a proximal portion of the elongated surgical device, the device holder comprising: a tube contact portion extending from a distal end of the device holder, the tube contact portion placed within the tube space and touching at least a portion of an internal surface of the tube, a handle portion extending from a proximal end of the device holder, and a housing cavity housing the proximal portion of the elongated surgical device; a removable cap engaged with the proximal end of the tube and having an internal space housing the handle portion of the device holder; and an end cap disposed at a distal end of the tube; removing the removable cap; pulling the device holder out of the tube by holding the handle portion of the device holder; opening the device holder to expose the proximal portion of the elongated surgical device; and removing the elongated surgical device from the device holder.

In some embodiments, the device holder further comprises a hinge; and the device holder is opened about the hinge. In some embodiments, the device holder further comprises a snap-fit mechanism; and the device holder is opened by sliding one portion of the device holder with respect to another portion, thereby disengaging the snap fit mechanism. In some embodiments, the handle portion of the device holder is exposed after the removable cap is removed. In some embodiments, the device holder is pulled out of the tube without touching the elongated surgical device. In some embodiments, the device holder is opened without touching the elongated surgical device.

Each feature or concept outlined above is independent and can be combined with other features or concepts outlined above or with any other feature or concept disclosed in this application.

### BRIEF DESCRIPTION OF THE DRAWINGS

FIG. 1 is a side elevational view of a packaging and an elongated surgical device within the packaging, in accordance with one embodiment of the technology described herein.

FIG. 2 illustrates opening of a removable cap from the packaging of FIG. 1.

FIG. 3 is a schematic view of threads of the removable cap and a tube for a design with a torturous path.

FIG. 4 is an enlarged view of a proximal portion of the packaging of FIG. 1.

FIG. 5 is an enlarged view of a proximal portion of the packaging of FIG. 1, without the removable cap.

FIG. 6 is a schematic view of a device holder and a portion of a surgical device, in accordance with one embodiment of the technology described herein, with the device holder partially opened.

FIG. 7 is a schematic view of the device holder of FIG. 6 and a portion of the surgical device of FIG. 6, with the device holder more fully opened.

FIG. 8 is a perspective view of a device holder, in accordance with another embodiment of the technology described herein

FIG. 9 illustrates the device holder of FIG. 8 in an opened state.

FIG. 10 is a perspective view of a device holder and a portion of a surgical device, in accordance with yet another embodiment of the technology described herein.

FIG. 11 is a cross-sectional view of the device holder of FIG. 10.

FIG. 12 is a perspective view of a device holder, in accordance with yet another embodiment of the technology described herein.

### DETAILED DESCRIPTION

### Packaging System

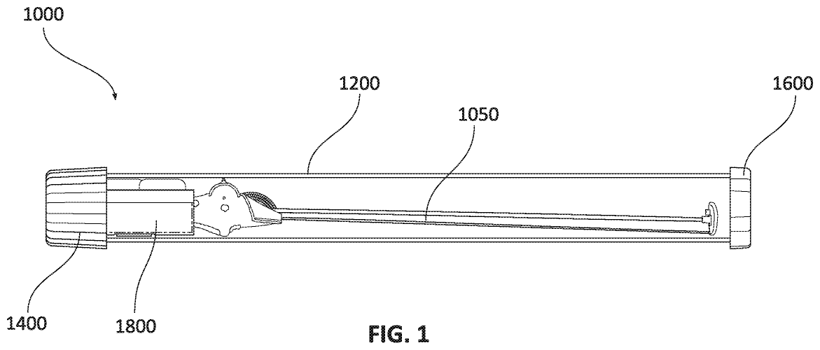

FIG. 1 illustrates a packaging system 1000 for a surgical device 1050. In some embodiments, as shown in FIG. 1, the packaging system 1000 includes a tube 1200, a removable cap 1400 positioned at one end of the tube 1200, an end cap 1600 positioned at the other end of the tube 2000, and a device holder 1800 for holding the device 1050. Each of these components will be described in greater detail with reference to the figures that follow.

### Surgical Device

A medical device described herein, such as the surgical device 1050, may be any surgical or medical device suitable for the packaging system 1000. In some embodiments, the medical device may have a generally elongated shape, extending from a proximal end to a distal end. In some embodiments, the medical device may be the surgical device 1050. In some embodiments, the surgical device 1050 has a generally elongated shape, extending from a proximal end to a distal end, and includes a handle at a proximal portion of the surgical device 1050. In some embodiments, the surgical device 1050 may be a catheter device.

### Tube

The tube 1200 may have a generally elongated shape, extending from a proximal end to a distal end. The tube 1200 may further include a tube space therein, such that at least a portion of the surgical device 1050 may be positioned within the tube. In some embodiments, the tube 1200 may have a cylindrical shape. In some embodiments, the tube 1200 may have a circle, square, pentagon, hexagon, or oval cross-sectional shape. The tube 1200 may have a sufficient width, length, or diameter such that the medical device can be packaged in the packaging system 1000 without touching an internal surface of the tube 1200. The tube 1200 may have a length and diameter sufficient to house the surgical device.

In some embodiments, at least a portion of the tube 1200 is transparent or translucent, such that the surgical device 1050 is visible when the surgical device 1050 is housed within the packaging system 1000. The tube 1200 may have sufficient strength to protect the surgical device 1050 from outside shock. The tube 1200 may be vapor impermeable/gas impermeable. The tube 1200 may have sufficient strength to protect the surgical device 1050 from outside shock. In some embodiments, the tube 1200 may be made from any suitable materials. For example, the tube 1200 may be made from polyethylene terephthalate (PET), polycarbonate (PC), polypropylene (PP), or another suitable material.

### Removable Cap

The removable cap 1400 may be coupled to the proximal end of the tube 1200, thereby closing the proximal end of the tube 1200. The removable cap 1400 may be removably and/or reversibly coupled to the proximal end of the tube 1200, such that the removable cap 1400 may be coupled and decoupled without significantly damaging the cap 1400 or the tube 1200.

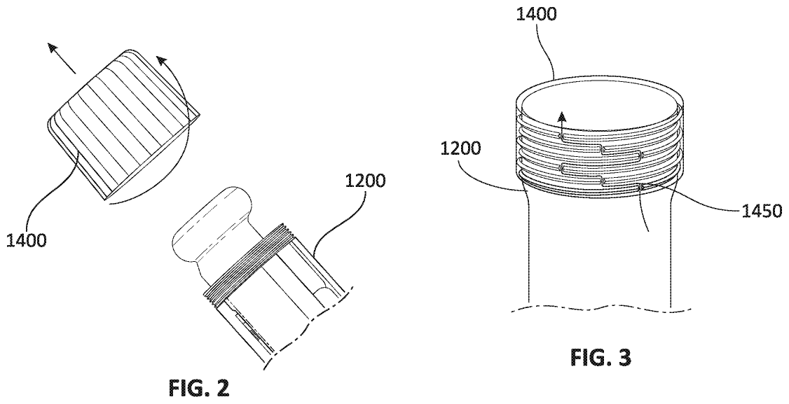

In some embodiments, the removable cap 1400 may be coupled to the proximal end of the tube 1200 by threads, such that the removable cap 1400 may be rotated about the tube 1200 to couple/decouple, as shown in FIG. 2. In some embodiments, an inner surface of the removable cap 1400 and/or the proximal end of the tube 1200 may be threaded. In other embodiments, the removable cap 1400 may be coupled to the proximal end of the tube 1200 by other known mechanisms in the art, such as friction fit.

In some embodiments, an outer surface of the removable cap 1400 may be shaped and/or textured to enhance the grip. For example, the removable cap 1400 may have a raised pattern, as shown in FIG. 2. In some embodiments, the removable cap 1400 may have a circular or substantially circular cross section. In some embodiments, the exterior surface of the removable cap 1400 may be angled to prevent rolling of the packaging 1000.

In some embodiments, the removable cap 1400 forms a sterile or substantially sterile seal when fully coupled with the tube 1200, such that the tube space and/or the surgical device 1050 is not contaminated. In some embodiments, the removable cap 1400 forms a bacterial barrier when fully coupled with the tube 1200. In some embodiments, the removable cap 1400 may allow air ingress/egress when coupled with the tube 1200. For example, the threads on the removable cap 1400 and/or the tube 1200 may form a tortuous path 1450, such that the assembly of the removable cap 1400 and the tube 1200 allows gas ingress/egress, while preventing microbes entering the tube space of the tube 1200. In other embodiments, the removable cap 1400 and the tube 1200 may form an air-tight seal. In some embodiments, the removable cap 1400 may include one or more gaskets or O-rings to form a seal with the tube 1200.

### Device Holder

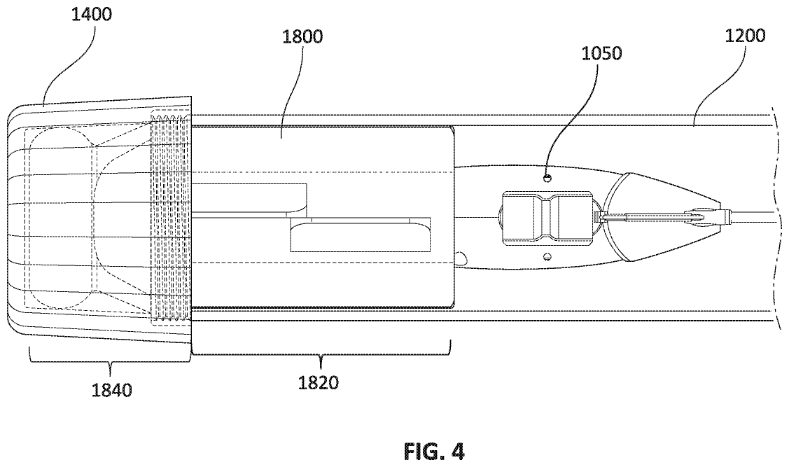

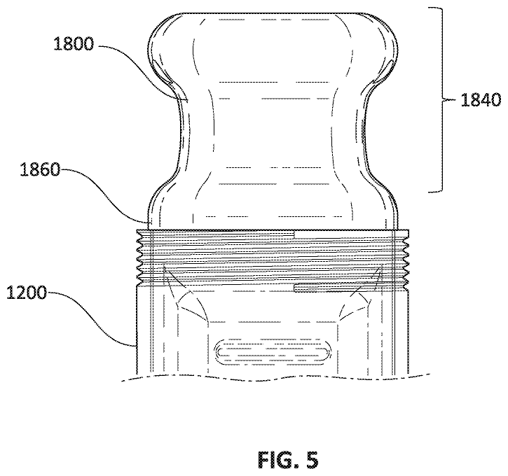

The device holder 1800 can releasably hold a proximal portion of the surgical device 1050, such that a user or clinician can take the surgical device 1050 from the tube 1200 without directly touching the surgical device 1050. FIGS. 4 and 5 illustrate the device holder 1800 according to one embodiment. As shown in FIGS. 4 and 5, the device holder 1800 may include a tube contact portion 1820 and a handle portion 1840. Further, the device holder 1800 is configured to hold the proximal portion of the surgical device 1050 such that the surgical device 1050 is suspended and does not touch the internal surface of the tube 1200. In some embodiments, the distal end or end tip of the surgical device 1050 does not touch the internal surface of the tube 1200.

As shown in FIG. 4, the tube contact portion 1820 may extend from a distal end or near the distal end of the device holder 1800, and is configured to be placed within the tube space of the tube 1200 and touch at least a portion of an internal surface of the tube 1200, when the packaging 1000 is assembled. As the tube contact portion 1820 touches the internal surface of the tube 1200, the device holder 1800 and the surgical device 1050 are prevented from moving within the packaging, and the surgical device 1050 can be kept suspended within the tube 1200.

Accordingly, as the tube contact portion 1820 is larger relative to the diameter of the tube 1200, the device holder 1800 and the surgical device 1050 will be more stably positioned within the tube. In some embodiments, the tube contact portion 1820 may have a length of at least 50%, 45%, 40%, 35%, 33.3%, 30%, 25%, 20%, 16.7%, 15%, 10%, or 5% of the length of the entire length of the tube 1200. In some embodiments, the tube contact portion 1820 may have a length of at least 50%, 45%, 40%, 35%, 33.3%, 30%, 25%, 20%, 16.7%, 15%, 10%, or 5% of the length of the entire length of the surgical device 1050. In some embodiments, the tube contact portion 1820 has a length which is at least 1.1, 1.2, 1.3, 1.4, 1.5, 1.6, 1.7, 1.8, 1.9, 2.0, 2.2, 2.4, 2.5, 2.6, 2.8, or 3.0 times the diameter of the surgical device 1050. In some embodiments, substantially the entire tube contact portion 1820 contacts the internal surface of the tube 1200. In some embodiments, only a portion of the tube contact portion 1820 contacts the internal surface of the tube 1200. In some embodiments, the tube contact portion 1820 may have a length of at least 50%, 45%, 40%, 35%, 33.3%, 30%, 25%, 20%, 16.7%, 15%, 10%, or 5% of the length of the entire length of the tube 1200. In some embodiments, the tube contact portion 1820 may have a length of at least 80%, 75%, 70%, 66.7%, 60%, 50%, 45%, 40%, 35%, 33.3%, 30%, 25%, 20%, 16.7%, 15%, 10%, or 5% of the length of the length of the device holder 1800.

In some embodiments, the tube contact portion 1820 may have a diameter substantially the same as the inner diameter of the tube 1200, such that the tube contact portion 1820 snuggly fits within the tube 1200. In some embodiments, the tube contact portion 1820 may have substantially the same cross-sectional shape as the tube space of the tube 1200, such that the tube contact portion 1820 snuggly fits within the tube 1200. For example, the tube contact portion 1820 may have a generally circular cross-sectional shape.

As shown in FIG. 5, the handle portion 1840 may extend from a proximal end or near the proximal end of the device holder 1800, and at least a portion of the handle portion 1840 is configured to be positioned outside the tube space of the tube 1200, such that a clinician or user can hold the handle portion 1840 and pull the assembly of the device holder 1800 and the surgical device 1050 out of the tube 1200. The handle portion 1840 may be shaped or processed to enhance the grip.

In some embodiments, the device holder 1800 includes a mechanism to prevent the device holder 1800 from entering excessively into the tube 1200. For example, as shown in FIG. 5, the device holder 1800 may include a ledge 1860 between the handle portion 1840 and the tube contact portion 1820, such that the handle portion 1840 cannot enter the tube space of the tube 1200. In some embodiments, at the ledge 1860, the device holder 1800 has a diameter greater than the inner diameter of the tube 1200. In some embodiments, at the ledge 1860, the device holder 1800 has a diameter smaller than the outer diameter of tube space of the tube 1200.

Turning back to FIG. 4, the removable cap 1400 may include an internal space that can house at least a portion of the handle portion 1840 of the device holder 1800. In some embodiments, the handle portion 1840 may contact at least a portion of the internal surface of the removable cap 1400, thereby providing a support to the device holder 1800 and the surgical device 1050, in addition to the support provided by the tube contact portion 1820 and the tube 1200. In some embodiments, substantially the entire handle portion 1840 may contact the internal surface of the removable cap 1400. In some embodiments, a portion of the handle portion 1840 may contact the internal surface of the removable cap 1400.

To hold the surgical device 1050, the device holder 1800 includes a housing cavity which can house the proximal portion of the surgical device 1050. As shown in FIG. 7, in some embodiments, the device holder 1800 has a housing cavity 1850 to house the proximal portion of the surgical device 1050. The housing cavity 1850 may have generally the same shape and size as the proximal portion of the surgical device 1050, such that the surgical device 1050 fits the device holder 1800 snugly, and such that the surgical device 1050 does not move about the housing cavity 1850, preventing undesired movement or wiggling of the surgical device 1050. In some embodiments, the proximal portion of the surgical device 1050 covered by the housing cavity 1850 may be at least 10%, 15%, 16.7%, 20%, 25%, 30%, 33.3%, 40%, or 50% of the length of the surgical device 1050. In some embodiments, at least a portion of a handle of the surgical device 1050 is held by the housing cavity 1850.

In some embodiments, the housing cavity 1850 may be shaped such that the surgical device 1050 cannot be released from the device holder 1800 by pulling it along the length of the surgical device. Such mechanism is useful for preventing accidental release of the surgical device 1050 from the device holder 1800. In some embodiments, the housing cavity 1850 and/or the surgical device 1050 may be tapered toward the distal direction, such that the surgical device 1050 cannot be released from the device holder 1800 by pulling it along the length of the surgical device.

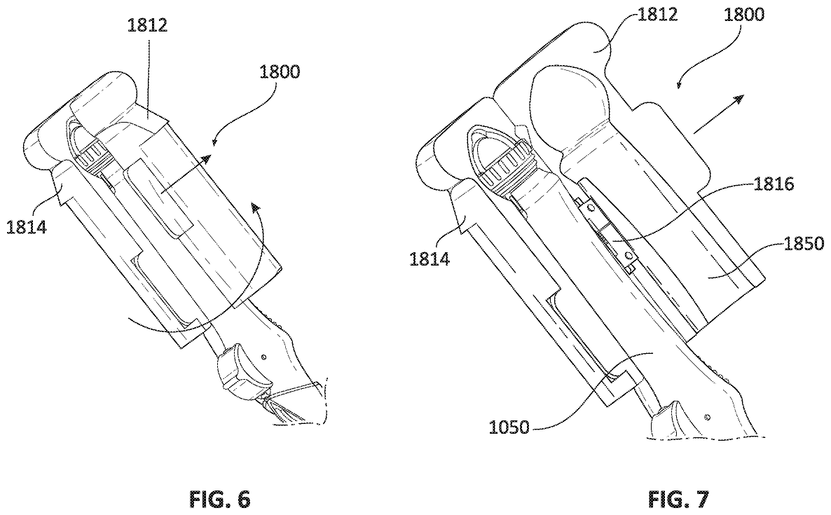

In some embodiments, the housing cavity 1850 is concealed within the device holder 1800, and the device holder 1800 has a plurality of divisions that can be separated and/or disassembled to expose the housing cavity 1850. The divisions may be disassembled to release the surgical device 1050, without complex steps, such that the surgical device can be released quickly, and thus without contamination in an operating room. In some embodiments, the device holder 1800 may be disassembled without touching the surgical device 1050. In some embodiments, the device holder 1800 includes a hinge connecting the divisions of the device holder 1800. FIGS. 6 and 7 illustrate an embodiment of the device holder 1800 having a hinge 1816. As shown in FIGS. 6 and 7, the device holder 1800 may have a clamshell-like design, and have two divisions 1812 and 1814. The two divisions are connected by the hinge 1816, such that the divisions 1812 and 1814 can move about the hinge 1816. The device holder 1800 may be opened about the hinge 1816 to expose the housing cavity 1850 and/or the surgical device 1050.

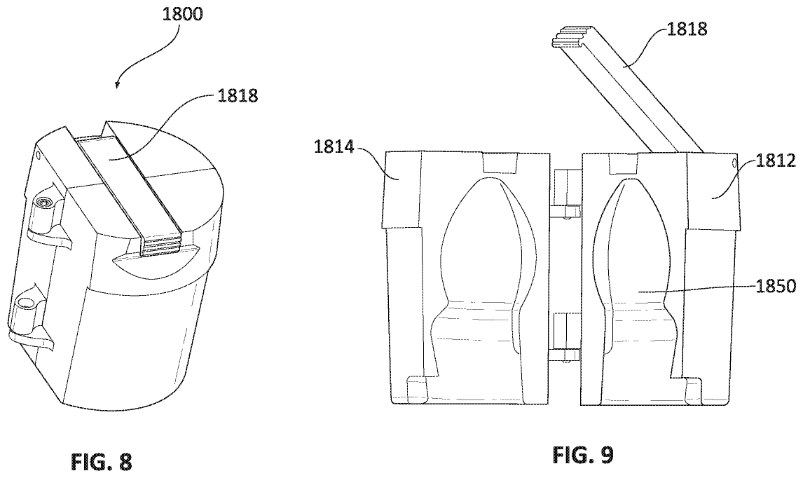

FIGS. 8 and 9 illustrate a device holder 1800 similar to the device holder of FIGS. 6 and 7. As shown in FIGS. 8 and 9, the device holder 1800 includes two divisions 1812 and 1814 and the hinge 1816. In some embodiments, the device holder 1800 may further include a locking element 1818, which prevents the opening of the device holder 1800 when locked, and allows the opening of the device holder 1800 when unlocked. In some embodiments, the locking element 1818 may extend along the groove across the two divisions 1812 and 1814 as shown in FIGS. 8 and 9. In some embodiments, the locking element 1818 may have any other forms known in the art. In some embodiments, the two divisions 1812 and 1814 may be releasably coupled to each other, for example by interlocking or friction fit, such that the device holder 1800 may be opened about the hinge 1816, by application of force.

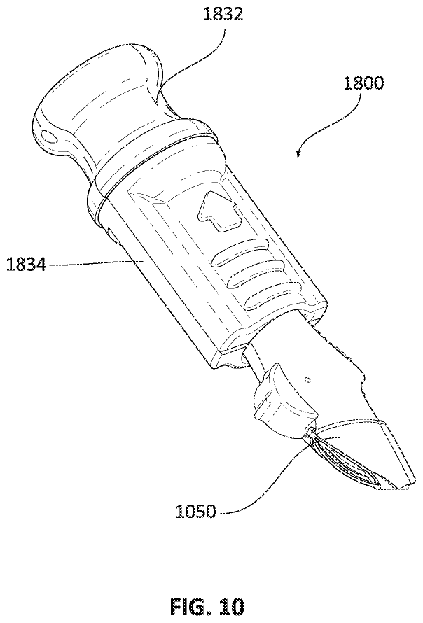

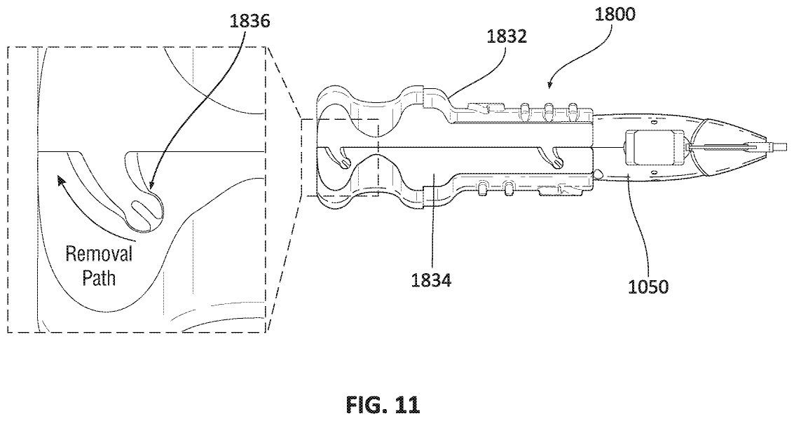

In some embodiments, the device holder 1800 may have two or more divisions which can be decoupled completely. In some embodiments, such as shown in FIG. 10, the device holder 1800 may include a first division 1832 and a second division 1834 connected via a snap fitting mechanism. For example, as shown in FIG. 11, the first division 1832 may include one or more protrusions 1836 to be coupled with the second division 1834, and the first division 1832 may be decoupled from the second division 1834 by sliding it in one direction. As shown in FIG. 10, the first division 1832 may include a sign (e.g., a arrow) to indicate the direction to slide it for removal.

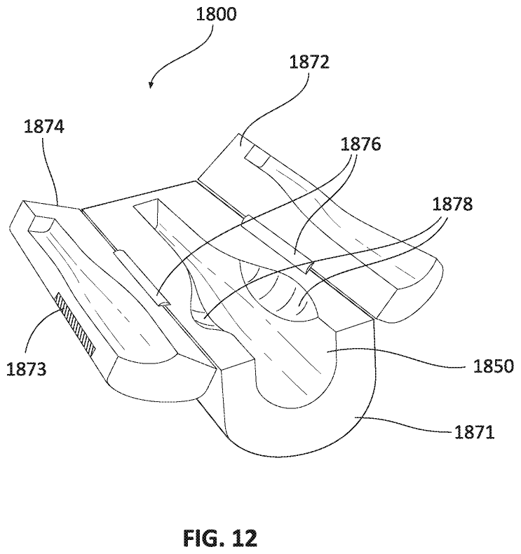

FIG. 12 illustrates another embodiment of the device holder 1800. In the illustrated embodiment, the device holder 1800 includes three divisions-a main division 1871, and two top divisions 1872 and 1874. The two top divisions 1872 and 1874 may each be connected to the main division 1871 through hinges 1876, such that the top divisions 1872 and 1874 can be opened to expose the housing cavity 1850. The device holder 1800 may include one or more recesses 1878 adjacent the housing cavity 1850, to assist grabbing the surgical device 1050. In some embodiments, the top divisions 1872 and 1874 may include a handling recess or groove 1873 to facilitate grabbing and opening of the divisions 1872 and 1874. In some embodiments, the top divisions 1872 and 1874 are symmetrical. In some embodiments, the main division 1871 may have a flat portion on its outer surface, such that the device holder, when opened, can be placed flat without rolling, exposing the housing cavity 1850 and/or the surgical device 1710.

### End Cap

Turning back to FIG. 1, the packaging 1000 includes an end cap 1600 at the distal end of the tube 1200. In some embodiments, the end cap 1600 forms an airtight seal with the tube 1200. In some embodiments, the end cap 1600 may be permanently attached to the tube 1200. In some embodiments, the end cap 1600 may be removably attached to the tube 1200. For example, the end cap 1600 may have threads, in a similar manner described with regard to the removable cap 1400. In some embodiments, the end cap 1600 has an outer diameter greater than the outer diameter of the tube 1200. In some embodiments, the end cap 1600 has the same or substantially the same outer diameter as the removable cap 1400. In some embodiments, the end cap 1600 may have a circular or substantially circular shape. In some embodiments, the end cap 1600 may have an angled or polygonal cross-sectional shape, such that rolling of the packaging 1000 is inhibited.

### Kit

In some embodiments, a kit including the surgical device 1050 and the packaging 1000 described herein is provided. In some embodiments, the surgical device 1050 may be included in the packaging 1000. In some embodiments, the surgical device 1050 may be fixed within the device holder 1800 within the packaging 1000. In some embodiments, the packaging 1000 may be filled with nitrogen or inert gas. In some embodiments, the kit may further include any other elements to maintain the desired condition within the packaging 1000. For example, the kit may include desiccant, humidity absorbent, or oxygen absorbent.

### Method of Use

In some embodiments, the surgical device 1050 may be provided within the packaging 1000 described herein. Upon use of the surgical device 1050, for example by a clinician, the surgical device 1050 may be taken out from the packaging 1000, and a method to do so may include: removing the removable cap 1400 from the tube 1200; pulling the device holder 1800, which is coupled to the surgical device 1050, out of the tube 1200; opening the device holder 1800 to expose the proximal portion of the surgical device 1050; and removing the surgical device 1050 from the device holder 1080.

In some embodiments, the removable cap 1400 may be removed form the tube 1200 by rotating the removable cap 1400 about the tube 1200. After removing the removable cap 1400, the proximal end, or the handle portion 1840 of the device holder 1800, may be exposed. The device holder 1800, holding the surgical device 1050, may be removed or pulled out of the tube 1200 by holding the handle portion 1840, such that the surgical device 1050 does not have to be touched, preventing contamination. In some embodiments, the assembly of the device holder 1800 and the surgical device 1050 may be placed on a sterile surface.

Subsequently, the device holder 1800 may be opened or disassembled to expose the proximal portion of the surgical device 1050. In some embodiments, the device holder 1800 comprises one or more hinges as described herein in relation to FIGS. 6 and 7, and the device holder 1800 is opened about these one or more hinges. In some embodiments, the device holder 1800 includes a snap-fit mechanism, as described herein in relation to FIG. 10, and the device holder 1800 is opened by sliding one portion of the device holder 1800 with respect to another portion, thereby disengaging the snap-fit mechanism. In some embodiments, the device holder 1800 may be opened without touching the surgical device 1050.

The packaging 1000 and/or the surgical device 1050 may be sterile or substantially sterile. In some embodiments, each of the packaging 1000 and the surgical device 1050 may be sterilized before assembling the packaging 1000. In some embodiments, the packaging 1000 and the surgical device 1050 may be sterilized after the packaging 1000 is assembled and the surgical device 1050 is contained within the surgical device 1050. In some embodiments, the packaging 1000, may be further packaged within a microbial barrier, such that the packaging 1000 may be kept sterilized during storage. The packaging 1000 and the surgical device 1050 may be sterilized by any suitable methods known in the art, such as heat, chemical, steam, and/or radiation.

### Definitions

It should be appreciated that some embodiments described in the foregoing description provide an improved packaging assembly featuring an interference force within a desired range over a larger range of interference widths, thereby securely maintaining a bioprosthetic heart valve within the packaging system, while also allowing easy removal of the heart valve from the packaging system without damage or contamination.

Specific methods, devices, and materials are described, although any methods and materials similar or equivalent to those described can be used in the practice or testing of the present examples. Unless defined otherwise, all technical and scientific terms used herein have the same meanings as commonly understood by one of ordinary skill in the art to which these examples belong.

The terms “a” and “an” encompass one or more of the specified elements. That is, if two of a particular element are present, one of these elements is also present and thus “an” element is present. The phrase “a plurality of” means two or more of the specified elements. The term “or” used between the last two of a list of elements means any one or more of the listed elements. For example, the phrase “A, B, or C” means “A, B, and/or C,” which means “A,” “B,” “C,” “A and B,” “A and C,” “B and C,” or “A, B, and C.” The term “coupled” generally means physically coupled or linked and does not exclude the presence of intermediate elements between the coupled items absent specific contrary language.

Without further elaboration, it is believed that the preceding description enables one skilled in the art to make and use the same to the fullest extent. The detailed description provides only the presently preferred examples. Persons skilled in the art will appreciate that various modifications can be made without departing from scope of the disclosure, which is defined only by the following claims.

What is claimed is:

1. A packaging for an elongated device, the packaging comprising:
a tube comprising a tube space therein;
a device holder configured to releasably hold a proximal portion of the elongated device, the device holder comprising:
a tube contact portion extending from a distal end of the device holder, the tube contact portion configured to be placed within the tube space and touch at least a portion of an internal surface of the tube,
a handle portion extending from a proximal end of the device holder,
a ledge between the tube contact portion and the handle portion, the ledge configured to rest on a proximal end of the tube such that the tube contact portion can be inserted into the tube space while the handle portion remains outside the tube, and
a housing cavity configured to house the proximal portion of the elongated device;
a removable cap having an internal space configured to house the handle portion of the device holder; and
an end cap disposed at a distal end of the tube;
wherein the removable cap is configured to be reversibly engaged with the proximal end of the tube; and
wherein, when the tube, the device holder, and the removable cap are assembled, the device holder is configured to hold the proximal portion of the elongated device such that the elongated device is suspended and does not touch the internal surface of the tube.

2. The packaging of claim 1, wherein the removable cap and the proximal end of the tube comprise threads.

3. The packaging of claim 2, wherein:
the removable cap is internally threaded; and
the proximal end of the tube is externally threaded.

4. The packaging of claim 1, wherein the removable cap and the tube are configured to form a microorganism-tight seal, such that microorganisms are inhibited from entering the tube space.

5. The packaging of claim 1, wherein the removable cap and the tube are configured to form a tortuous path, such that gas is allowed to ingress to or egress from the tube space via the tortuous path, while microorganisms are inhibited from entering the tube space.

6. The packaging of claim 1, wherein the packaging further comprises a gasket or an O-ring.

7. The packaging of claim 1, wherein the device holder has a plurality of divisions that are configured to be separated to expose the housing cavity.

8. The packaging of claim 7, wherein the device holder further comprises a hinge.

9. The packaging of claim 1, wherein the device holder is configured to contact at least a portion of an internal surface of the removable cap.

10. The packaging of claim 1, wherein:
the device holder has a length; and
the tube contact portion extends at least half of the length of the device holder.

11. The packaging of claim 1, wherein the ledge is configured to prevent the handle portion from entering the tube space.

12. The packaging of claim 1, wherein the tube contact portion has a length which is at least 20% of the length of the tube.

13. The packaging of claim 1, wherein:
the elongated device has a diameter; and
the tube contact portion has a length which is at least 1.5 times of the diameter of the elongated device.

14. A kit comprising the packaging of claim 1 and an elongated surgical device and wherein the elongated device is a catheter device.

15. A method of deploying an elongated surgical device, the method comprising:
providing the elongated surgical device contained in a packaging, wherein the packaging comprises:
a tube comprising a tube space therein;
a device holder releasably holding a proximal portion of the elongated surgical device, the device holder comprising:
a tube contact portion extending from a distal end of the device holder, the tube contact portion placed within the tube space and touching at least a portion of an internal surface of the tube,
a handle portion extending from a proximal end of the device holder, and
a housing cavity housing the proximal portion of the elongated surgical device;
a removable cap engaged with the proximal end of the tube and having an internal space housing the handle portion of the device holder; and
an end cap disposed at a distal end of the tube;
removing the removable cap;
pulling the device holder out of the tube by holding the handle portion of the device holder;
opening the device holder to expose the proximal portion of the elongated surgical device; and
removing the elongated surgical device from the device holder.

16. The method of claim 15, wherein:
the device holder further comprises a hinge; and
the device holder is opened about the hinge.

17. The method of claim 15, wherein:
the device holder further comprises a snap-fit mechanism; and
the device holder is opened by sliding one portion of the device holder with respect to another portion, thereby disengaging the snap-fit mechanism.

18. The method of claim 15, wherein the handle portion of the device holder is exposed after the removable cap is removed.

19. The method of claim 15, wherein the device holder is pulled out of the tube without touching the elongated surgical device.

20. The method of claim 15, wherein the device holder is opened without touching the elongated surgical device.
